(19) United States
(12) Patent Application Publication
(10) Pub. No.: US 2018/0155664 A1
(43) Pub. Date: Jun. 7, 2018

(54) MULTI-MICROWELL ARRAY FOR MULTI-CELLULAR ADJACENT CO-CULTURE

(71) Applicants: Maribella Domenech, San German, PR (US); Karla P. Ramos, Saint Just, PR (US)

(72) Inventors: Maribella Domenech, San German, PR (US); Karla P. Ramos, Saint Just, PR (US)

(21) Appl. No.: 15/431,683

(22) Filed: Feb. 13, 2017

Related U.S. Application Data
(60) Provisional application No. 62/293,836, filed on Feb. 11, 2016.

(51) Int. Cl. C12M 1/32; C12M 3/00

(52) U.S. Cl. CPC C12M 23/12; C12M 21/08

(57) ABSTRACT

The invention provides a multi-microwell platform that enables co-culture of up to 4 cell types in discrete adjacent wells using either 2-dimension or 3-dimension culture conditions for up to 10 days. The microwells are made of poly-dimethyl siloxane (PDMS) and placed on top of glass or tissue culture plastic. Cells and culture media can be manually loaded and replaced using a pipette independently of passive pumping method, and without the need of vacuum-assisted filling or syringe pumps. The major advantages of the inventive multi-microwell platform are reduction in cell number required as compared to well-based culture platforms, compatibility with manual pipetting, well-readout instruments and ease of use as compared to other microfluidic approaches, cell migration limited which doesn't requires cell labeling or sorting for cell-type specific studies, and adjacent multi-cellular 2-D and 3-D culture conditions which is in contrast with current methods where studies with limited cell numbers among multiple cell types are difficult or impossible to perform.

### BACKGROUND OF THE INVENTION

The need to examine multi-cellular interactions in vitro represents a significant challenge to furthering our understanding of basic mechanisms of cell regulation in biology. The ability to mix and match cells in culture devices enables the evaluation of the effect of multi-cellular signaling in cellular processes (e.g. growth, migration) providing tools for elucidating the relative roles each cell type plays and developing new strategies that target such roles. Few in vitro tools allow to probe intercell network communication (e.g. paracrine signals among multiple cell types) and those suitable for multiple cell studies lack of tunable compartments needed for cell type-specific studies of cell signaling and require high numbers of cells limiting studies to cell lines and excluding heterogeneous patient samples. Well-based technology puts cells into a high-volume environment that can sweep soluble signals away from their targets, diluting local concentrations. Transwell and matrix-embedded co-culture systems enable co-culture of two adjacent cell types but have limited optics compatibility in z-axis, long diffusion distances that exclude short range secreted factors, require high numbers of cells limiting the studies to cell lines and excluding heterogeneous patient samples, and are restricted to two compartments limiting the study of cell signaling to just two cell types. Low volume well plates, such as Terasaki plates, can be used to increase cell volume ratio and the culture small amount of cells but are limited only to one cell type. Mixing of more than two cell types within the same culture compartment requires multi-cell labeling, specialized cell-sorting instruments and trained personnel which are not available in most biology laboratories and undergraduate research institutions. Thus, systems that provide a controlled and defined culture environment were multiple cell types can interact in close proximity amenable to routine used in laboratories are needed to understand the mechanisms by which multi-cellular communication promotes tumorigenesis.

Microscale technology can overcome some of the limitations of current culture platforms by providing tunable compartments needed for cell signaling studies that allow precisely control spatial and temporal dose exposure, use of high-throughput formats (e.g. 96 well plates) compatible with existing microscopy and reporter-based endpoints. Previous studies demonstrated that microchannel culture enhances communication among multiple cell types by providing a controlled and defined culture environment where multiple cell types can interact in close proximity, and in which secreted factors accumulate faster than in convectional wells. Microscale devices have also show significant advances in clinical research. Co-culture and migration studies in multi-compartmentalize micro-devices have mimicked cellular interactions observed at the tissue level providing a platform in which to study the molecular biology of diseases at the multi-cellular level and evaluate the efficacy of potential drug candidates and strategies for clinical application. Although, at this point microscale devices do not replace animal studies, they provide a unique environment in which multi-cellular interactions can be monitored and cell-specific information can be collected at multiple time points. Thus, microscale devices enable observations at the multi-cellular level that are not possible with current platforms allowing the formulation of new hypotheses that can lead to the discovery of potential clinical therapies.

### SUMMARY OF THE INVENTION

The present invention characterized a multi-microwell platform that enables co-culture of up to 4 cell types in discrete adjacent wells using either 2-dimension or 3-dimension culture conditions for up to 10 days.

According to an aspect of the invention, the microwells are made of poly-dimethyl siloxane (PDMS) and placed on top of glass or tissue culture plastic.

According to another aspect of the invention, cells and culture media can be manually loaded and replaced using a pipette independently of passive pumping method, and without the need of vacuum-assisted filling or syringe pumps.

According to yet another aspect of the invention, the cell culture surface area of each microwell is 5.7 mm² and can accommodate 5,000-10,000 cells.

According to still another aspect of the invention, the design of each microwell can be scale-down to accommodate smaller cell samples.

In accordance to an aspect of the invention, cell paracrine signaling was evaluated to show the feasibility of this platform for multi-cell soluble factor signaling studies.

According to another aspect of the invention, the multi-microwell platform provides a reduction in the cell number required as compared to well-based culture platforms, compatibility with manual pipetting, well-readout instruments and ease of use as compared to other microfluidic approaches.

According to yet another aspect of the invention, the cell migration is limited which doesn't requires cell labeling or sorting for cell-type specific studies.

According to one aspect of the invention, adjacent multi-cellular 2-D and 3-D culture conditions are provided which is in contrast with current methods where studies with limited cell numbers among multiple cell types are difficult or impossible to perform.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Further features and advantages of the invention will become apparent from the following detailed description taken in conjunction with the accompanying figures showing illustrative embodiments of the invention, in which:

FIG. 1 illustrates PDMS microwells for multi-cell culture, according to the present invention.

FIG. 2 shows images of a SU-8 silicon oxide wafer master mold and resulting PDMS microwell array aligned to a 96 well plate format, according to the present invention.

FIG. 3A shows an image of a combination of 2 sizes of multi-micro well array of 3 adjacent wells, according to the present invention.

FIG. 3B shows an image of 3D co-culture of fibroblasts and cancer cells embedded in collagen matrix, according to the present invention.

FIG. 3C shows an image of a viability assay of Induced Pluripotent Cardiomyocytes embedded in collagen matrix after 24 hrs of culture, according to the present invention.

### DETAIL DESCRIPTION OF THE INVENTION

### Methods

### Microfabrication of Multi-Microwell Array

The microwell mask was designed with Adobe illustrator and fabricated using convectional SU-8 photolithography fabrication methods (D. C. Duffy, et al., Rapid prototyping of microfluidic systems in Poly-dimethylsiloxane). The microwells have a diameter of 2.7 mm and height of 250 μm. The outer ring designed to contain the cell culture media drop that connects the microwells has a diameter of 8 mm and height of 750 μm (from tissue culture plastic bottom). Microwell array was fabricated using well-described soft lithography methods (Y. N. Xia and G. M. Whitesides, Soft lithography, Angew. Chem., Int. Ed., 1998). Briefly, PDMS and crosslinker are mixed manually at a 10:1 ratio, degassed under vacuum, poured over an SU-8 master mold and cured for 6 hrs at 60° C. The PDMS micro-well array is peeled-off from the master and placed in 95% ethanol for 12-24 hrs to remove un-crosslinked oligomers. PDMS micro-well array is sterilized for 20 min under ultraviolet light in the biosafety cabinet and mounted on top of a flat-bottom tissue-culture treated plate (Nunc, Rochester, N.Y.) or microscope glass slide for cell attachment. A 10 ul volume is used to seed cells in each microwell and 50 ul drop is used to connect the microwells. An amount of 4 ml volume is placed around the edge of PDMS array and the trays are sealed with paraffin to prevent evaporation.

### Cell Culture:

MDA-MB-231 and NIH-3T3 were purchased from American Tissue culture Collection (ATCC). All cells were maintained in DMEM high glucose media (Invitrogen), supplemented with 10% heat inactivated fetal bovine serum (Gibco), 1% amphotericin B and 1% penicillin/streptomycin (complete cell culture medium) at 37C in 5% CO₂. Cells were passaged with 0.05% trypsin-EDTA solution (Invitrogen) when grown near 75-80% confluence. Viable cells were counted using a hemocytometer (0.4% Trypan Blue solution), diluted to the desired cell densities and seeded in microwells.

### Results

### Characterization of a PDMS-Based Multi-Microwell Array

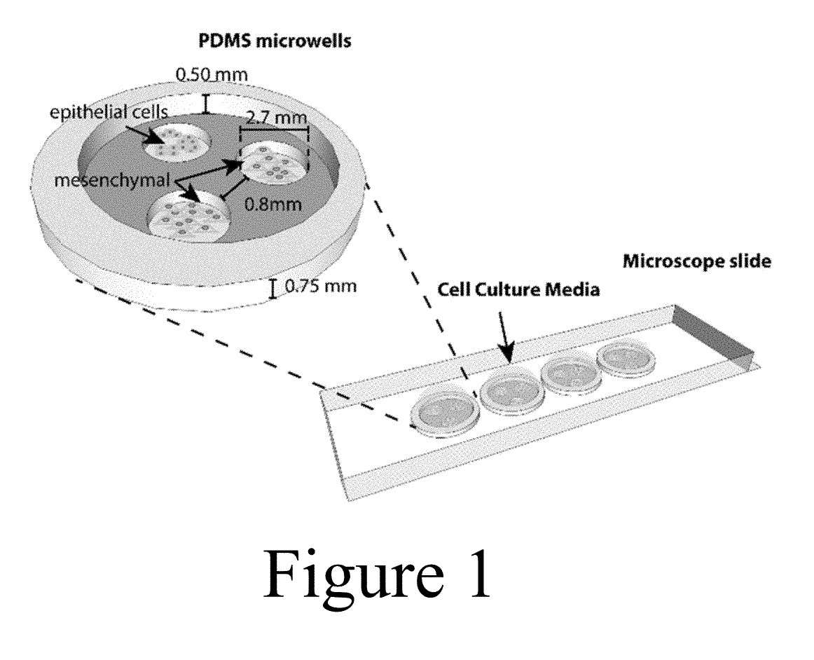

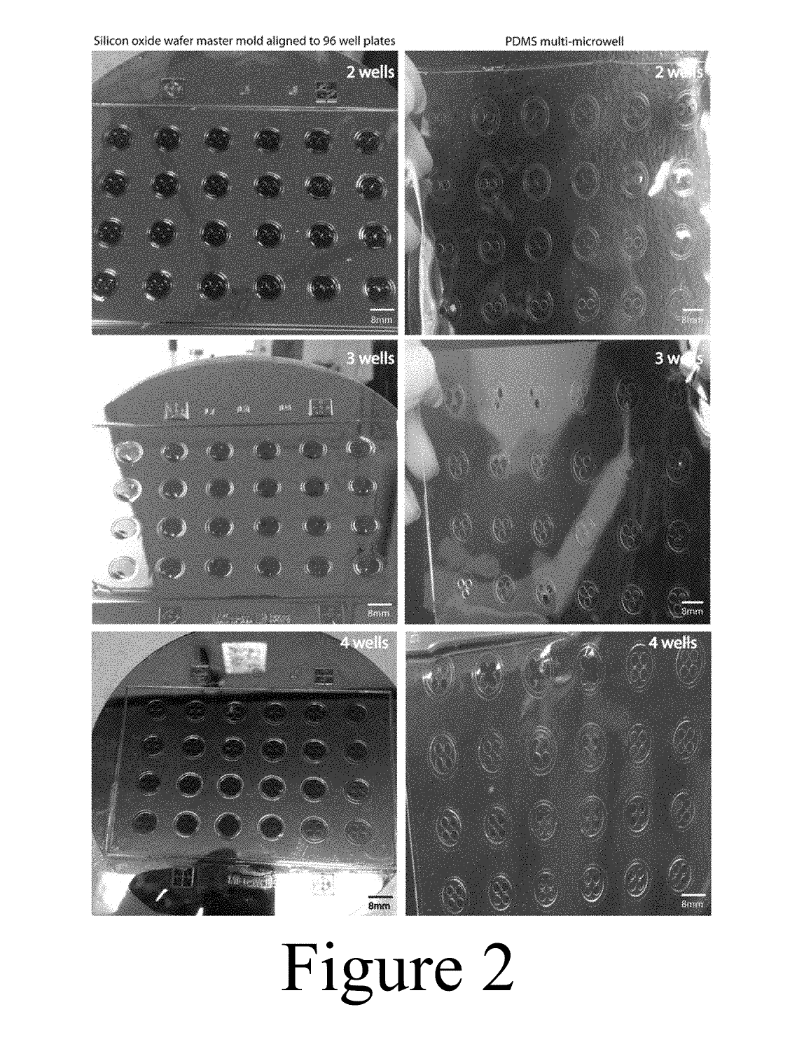

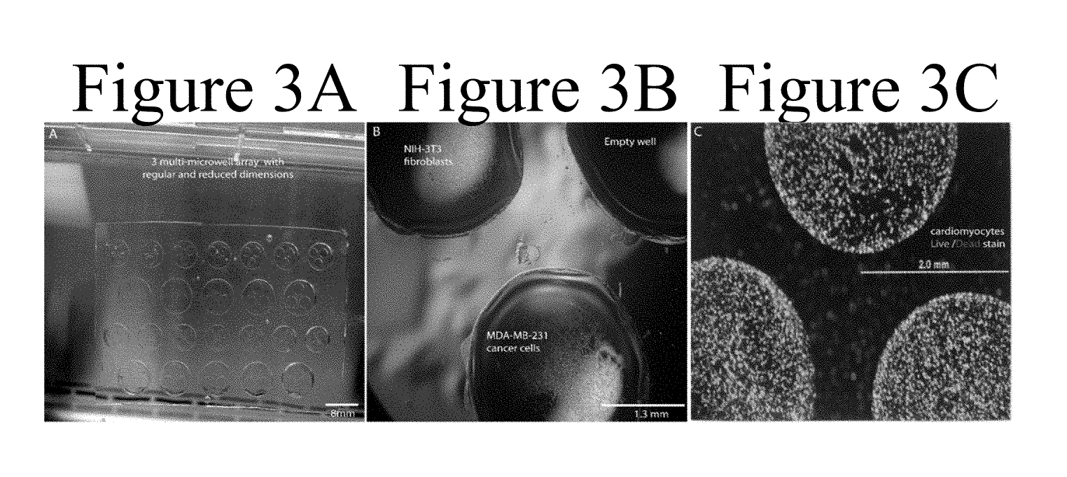

The multi-microwell culture array consists of one to four adjacent wells (3-microwell is shown in FIG. 1, where PDMS is shown in dark and light gray colors) contained within a bigger well. A simple circular geometry of open PDMS micro-wells allows for cell seeding in adjacent yet independent compartments that can be connected by dispensing a ˜50 ul cell culture drop above them. The microwells have a diameter of 2.7 mm and height of 200 um. The microwells are contained within an outer-ring of 8 mm diameter and 750 um height. Each microwell was loaded independently by dispensing 10 ul volume using a manual pipette. This allows cells to attach prior to co-culture. Computer simulation results show that the microwell volume allows for diffusion-based communication between the microwells within the relevant experimental times. The PDMS multi-microwell array can have 2-4 wells adjacent for independent seeding of 4 cell types and aligned to a 96 well plate format for readouts in standard optical instruments, as shown in FIG. 2. Microwells can be scale down for reduced cell sample as shown in FIG. 3A. Cells can be cultured adjacent in 3 dimensions and remain viable for 24 hrs as shown in FIGS. 3B and 3C, where live cells are shown in green and dead cells are shown in red. Other experiments have shown that attached cells remained viable for up to 7 days. Cell viability (monitored at 24, 48, 96 and 168 hours) was maintained above 80% up to 168 hours with half media replacements every 48 hrs.

1) A multi-microwell culture array comprising:
a microwell-containing element including a plurality of adjacent and discrete circular microwells and an outer ring extending upwardly from an upper surface of said plurality of adjacent and separated circular microwells.

2) The multi-microwell culture array of claim 1, further comprising cell culture medium dropped over said microwell-containing element and being contained by said outer ring.

3) The multi-microwell culture array of claim 1, wherein said microwell-containing element is positioned over of a tissue culture material.

4) The multi-microwell culture array of claim 1, wherein tissue culture material comprises at least one of: glass or tissue culture plastic.

5) The multi-microwell culture array of claim 1, wherein cells and culture media is manually loaded and replaced inside said plurality of microwells.

6) The multi-microwell culture array of claim 5, wherein the media is manually loaded with a pipette.

7) The multi-microwell culture array of claim 1, wherein a cell culture surface area of each microwell is 5.7 mm².

8) The multi-microwell culture array of claim 1, wherein each microwell accommodates 5,000-10,000 cells.

9) The multi-microwell culture array of claim 1, wherein each microwell has a diameter of 2.7 mm and a height of 200 μm.

10) The multi-microwell culture array of claim 1, wherein said outer ring has a diameter of 8 mm and a height of 750 μm.

11) The multi-microwell culture array of claim 1, wherein the size of each microwell is selected to accommodate different sized cell samples.

12) The multi-microwell culture array of claim 1, wherein said adjacent and discrete circular microwells allow 2-Dimensional and 3-Dimensional culture of at least one cell type without mixing.

13) The multi-microwell culture array of claim 1, wherein said adjacent and discrete circular microwells allow 2-Dimensional and 3-Dimensional culture of at least two different cell types without mixing.

14) The multi-microwell culture array of claim 2, wherein the cell culture medium allows connection within said plurality of microwells.

15) A well-based culture plate comprising a plurality of the multi-microwell culture array of claim 1.
